(19) United States
(12) Patent Application Publication
(10) Pub. No.: US 2005/0095599 A1
(43) Pub. Date: May 5, 2005

(54) Detection and identification of biopolymers using fluorescence quenching

(76) Inventors: Richard J. Pittaro, San Carlos, CA (US); Edward D. Verdonk, San Jose, CA (US); Thomas P. Doherty, San Mateo, CA (US)

Correspondence Address: AGILENT TECHNOLOGIES, INC.;Legal Department, DL429, Intellectual Property Administration, P.O. Box 7599, Loveland, CO, 80537-0599, US

(21) Appl. No.: 10/699,478

(22) Filed: Oct. 30, 2003

(51) Int. Cl. C12Q 1/68; G01N 33/53; C12M 1/34

(52) U.S. Cl. 435/6; 435/7.1; 435/287.2

(57) ABSTRACT

The present invention provides an apparatus for detecting a nanoscale moiety and a method for sensing a nanoscale moiety. The apparatus includes a substrate having a nanopore, at least one excitable molecule attached to the substrate adjacent to the nanopore, and a light source for exciting the excitable molecule attached to the substrate adjacent to the nanopore wherein the excitable molecule is quenched by the quencher molecule on the nanoscale moiety as it passes by the excitable molecule. The invention also includes a method for detecting the presence or identity of the nanoscale moiety.

### TECHNICAL FIELD

The invention relates generally to the field of biopolymers and more particularly to an apparatus and method for identifying and characterizing biopolymer molecules.

### BACKGROUND

Techniques for manipulating matter at the nanometer scale (“nanoscale”) are important for many electronic, chemical and biological purposes (See Li et al., “Ion beam sculpting at nanometer length scales”, Nature, 412: 166-169, 2001). Among such purposes are the desire to more quickly sequence biopolymers such as DNA. Nanopores, both naturally occurring and artificially fabricated, have recently attracted the interest of molecular biologists and biochemists for the purpose of DNA sequencing.

It has been demonstrated that a voltage gradient can drive a biopolymer such as single-stranded DNA (ssDNA) in an aqueous ionic solution through a naturally occurring transsubstrate channel, or “nanopore,” such as an α-hemolysin pore in a lipid bilayer. (See Kasianowicz et al., “Characterization of individual polynucleotide molecules using a membrane channel”, Proc. Natl. Acad. Sci. USA, 93: 13770-13773, 1996). The process in which the DNA molecule goes through the pore has been dubbed “translocation”. During the translocation process, the extended biopolymer molecule blocks a substantial portion of the otherwise open nanopore channel. This blockage decreases the ionic electrical current flow occurring through the nanopore in the ionic solution. The passage of a single biopolymer molecule can, therefore, be monitored by recording the translocation duration and the decrease in current. Many such events occurring sequentially through a single nanopore provide data that can be plotted to yield useful information concerning the structure of the biopolymer molecule. For example, given uniformly controlled translocation conditions, the length of the individual biopolymer can be estimated from the translocation time.

One desire of scientists is that the individual monomers of the biopolymer strand might be identified via the characteristics of the blockage current, but this hope may be unrealized because of first-principle signal-to-noise limitations and because the naturally occurring nanopore is thick enough that several monomers of the biopolymer are present in the nanopore simultaneously.

More recent research has focused on fabricating artificial nanopores. Ion beam sculpting using a diffuse beam of low-energy argon ions has been used to fabricate nanopores in thin insulating substrates of materials such as silicon nitride (See Li et al., “Ion beam sculpting at nanometer length scales”, Nature, 412: 166-169, 2001). Double-stranded DNA (dsDNA) has been passed through these artificial nanopores in a manner similar to that used to pass ssDNA through naturally occurring nanopores. Current blockage data obtained with dsDNA is reminiscent of ionic current blockages observed when ssDNA is translocated through the channel formed by α-hemolysin in a lipid bilayer. The duration of these blockages has been on the millisecond scale and current reductions have been to 88% of the open-pore value. This is commensurate with translocation of a rod-like molecule whose cross-sectional area is 3-4 nm² (See Li et al., “Ion beam sculpting at nanometer length scales”, Nature, 412: 166-169, 2001). However, as is the case with single-stranded biopolymers passing through naturally occurring nanopores, first-principle signal-to-noise considerations make it difficult or impossible to obtain information on the individual monomers in the biopolymer.

A second approach has been suggested for detecting a biopolymer translocating a nanopore in a rigid substrate material such as Si₃N₄. This approach entails placing two tunneling electrodes at the periphery of one end of the nanopore and monitoring tunneling current from one electrode, across the biopolymer, to the other electrode. However, it is well known that the tunneling current has an exponential dependence upon the height and width of the quantum mechanical potential barrier to the tunneling process. This dependence implies an extreme sensitivity to the precise location in the nanopore of the translocating molecule. Both steric attributes and physical proximity to the tunneling electrode could cause changes in the magnitude of the tunneling current which would be far in excess of the innate differences expected between different monomers under ideal conditions. For this reason, it is difficult to expect this simple tunneling configuration to provide the specificity required to perform biopolymer sequencing.

Resonant tunneling effects have been employed in various semiconductor devices including diodes and transistors. For instance, U.S. Pat. No. 5,504,347, Javanovic, et al., discloses a lateral tunneling diode having gated electrodes aligned with a tunneling barrier. The band structures for a resonant tunneling diode are described wherein a quantum dot is situated between two conductors, with symmetrical quantum barriers on either side of the quantum dot. The resonant tunneling diode may be biased at a voltage level whereby an energy level in the quantum dot matches the conduction band energy in one of the conductors. In this situation the tunneling current between the two conductors versus applied voltage is at a local maximum. At some other bias voltage level, no energy level in the quantum dot matches the conduction band energy in either of the conductors and the current versus applied voltage is at a local minimum. The resonant tunneling diode structure can thus be used to sense the band structure of energy levels within the quantum dot via the method of applying different voltage biases and sensing the resulting current levels at each of the different voltage biases. The different applied voltage biases can form a continuous sweep of voltage levels, and the sensed resulting current levels can form a continuous sweep of current levels. The slope of the current versus voltage can in some cases be negative. Conceptually, it is also possible to inject a known current between the conductors and measure the resulting voltage, but this approach can fail if the characteristic current versus voltage has a negative slope region. For this reason, applying a known voltage bias and sensing the resultant current is usually the preferred method.

The problem with many of these techniques regards the ability to actually obtain measurements from the biopolymers that translocate through nanopores. Theoretically, these systems should be capable of detecting and recording information that can distinguish one monomer from another. However, to date no concrete experimental data exists to show that this is actually possible. Therefore, there is a need for alternate systems and methods for identifying, detecting and characterizing biopolmers. In addition, there is a need for a system or method that may record and capture information traversing nanopores on a time scale of less than a microsecond. A number of techniques and systems have been employed for probing molecules on rapid time scales using fluorescence, phosphorescence or bioluminescense. These techniques often employ the use of a fluorophore or chromophore in a protein and a quencher molecule. A number of quencher molecules have been identified for probing protein and nucleic acids structures. For instance, some known quenchers include coumarin, fluorescein, cesium chloride, potassium iodide, oxygen, and quinaldic acid. Chromophores in proteins include aromatics amino acids such as tryptophan, phenylalanine, tyrosine and histidine. In nucleic acids, a number of studies have been conducted using guanine as a fluorophore.

The problem with many phosphorescence or fluorescence techniques is that they become rather difficult to control how and when a quencher molecule contacts a fluorophore or chromophore. In addition, for collisional quenching to take place the actual molecules need to contact or come within close proximity. In some systems that use chromophores, the excited molecules have been shown to transfer energy from the excited molecule to another molecule close by or in the vicinity. For instance, studies have been conducted using metals such as lanthanum or terbium to bind to calcium binding loops of proteins (EF hand calcium binding loop). The chromophore can then be excited and energy can be transferred to the metals from the chromophore by an energy transfer process. Both Dexter and Forster energy transfer models describe these energy transfer processes for different fluorophore to quencher distances. Energy transfer is contingent upon the proximity of the metal to the chromophore in the molecule. A resultant energy is emitted from the metals at defined wavelengths that are characteristic of the structure of the biomolecule. In other words, both excitation and emission spectra can be developed that show varying line shapes that are characteristic of a particular biomolecule.

The references cited in this application infra and supra, are hereby incorporated in this application by reference. However, cited references or art are not admitted to be prior art to this application.

### SUMMARY OF THE INVENTION

The invention provides an apparatus and method for detection and characterization of a nanoscale moiety such as a biopolymer. The invention provides a nanopore structure for sensing a nanoscale moiety. The nanopore structure comprises a substrate having a nanopore, an excitable molecule attached to the substrate adjacent to the nanopore and a light source for exciting the excitable molecule attached to the substrate adjacent to the nanopore, wherein the excitable molecule provides a first excitation signal capable of being quenched by a quencher molecule on the nanoscale moiety as it passes by the excitable molecule and the identify of the nanoscale moiety is capable of determination.

The invention also provides a method for sensing a portion of a nanoscale moiety. The method comprises providing a substrate having an excitable molecule adjacent to a nanopore and moving a portion of a nanoscale moiety with a quencher molecule past the excitable molecule to quench the excitable molecule and determine the presence and/or identity of the portion of the nanoscale moiety.

### BRIEF DESCRIPTION OF THE FIGURES

The invention is described in detail below with reference to the following figures:

FIG. 1A illustrates a schematic representation of an embodiment of the present invention.

FIG. 1B illustrates a second embodiment of the present invention.

FIG. 2A illustrates a third embodiment of the present invention.

FIG. 2B illustrates a fourth embodiment of the present invention.

FIG. 3 illustrates a theoretical stochastic sensing pattern produced from the modulation of the first excitation signal of the present invention.

### DETAILED DESCRIPTION OF THE INVENTION

This invention is not limited to specific compositions, methods, steps, or equipment, as such may vary. The terminology used herein is for the purpose of describing particular embodiments only, and is not intended to be limiting. Methods recited herein may be carried out in any order of the recited events that is logically possible, as well as the recited order of events. Furthermore, where a range of values is provided, it is understood that every intervening value, between the first and second limit of that range and any other stated or intervening value in that stated range is encompassed within the invention. Also, it is contemplated that any optional feature of the inventive variations described may be set forth and claimed independently, or in combination with any one or more of the features described herein.

Unless defined otherwise below, all technical and scientific terms used herein have the same meaning as commonly understood by one of ordinary skill in the art to which this invention belongs. Still, certain elements are defined herein for the sake of clarity. In the event that terms in this application are in conflict with the usage of ordinary skill in the art, the usage herein shall be controlling.

Where a range of values is provided, it is understood that each intervening value, to the tenth of the unit of the second limit unless the context clearly dictates otherwise, between the first and second limit of that range, and any other stated or intervening value in that stated range, is encompassed within the invention. The first and second limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used in this specification and the appended claims, the singular forms “a”, “an” and “the” include plural referents unless the context clearly dictates otherwise. Thus, for example, reference to “a biopolymer” includes more than one biopolymer, and reference to “a voltage source” includes a plurality of voltage sources and the like. In describing and claiming the present invention, the following terminology will be used in accordance with the definitions set out below.

A “biopolymer” is a polymer of one or more types of repeating units. Biopolymers are typically found in biological systems and particularly include polysaccharides (such as carbohydrates), peptides (which term is used to include polypeptides and proteins), glycans, proteoglycans, edgeids, sphingoedgeids, known biologicals materials such as antibodies, etc., and polynucleotides as well as their analogs such as those compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in hydrogen bonding interactions, such as Watson-Crick type, Wobble type and the like. Polynucleotides include single or multiple stranded configurations, where one or more of the strands may or may not be completely aligned with another. A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. Biopolymers include DNA (including cDNA), RNA, oligonucleotides, and PNA and other polynucleotides as described in U.S. Pat. No. 5,948,902 and references cited therein (all of which are also incorporated herein by reference), regardless of the source. An “oligonucleotide” generally refers to a nucleotide multimer of about 10 to 100 nucleotides in length, while a “polynucleotide” includes a nucleotide multimer having any number of nucleotides. A “biomonomer” references a single unit, which can be linked with the same or other biomonomers to form a biopolymer (e.g., a single amino acid or nucleotide with two linking groups one or both of which may have removable protecting groups).

An “excitable molecule” is any molecule that may transition from ground state to singlet or triplet state and then back to ground state. An excitable molecule may comprise an aromatic or multiple conjugated double bonds with a high degree of resonance stability. These classes of substances have delocalized π electrons that can be placed in low lying excited singlet states. In addition, these molecules may also comprise quantum dots or other molecules capable of absorbing and/or releasing energy. Quantum dots also have the advantage of not photo-bleaching. The excitable molecule may comprise one or more different dyes, quantum dots or any other molecules capable of absorbing and/or releasing energy.

A “substrate” refers to any surface that may or may not be solid and which is capable of holding, embedding, attaching or which may comprise the whole or portions of an excitable molecule.

“Hybridizing”, “annealing” and “binding”, with respect to polynucleotides, are used interchangeably. “Binding efficiency” refers to the productivity of a binding reaction, measured as either the absolute or relative yield of binding product formed under a given set of conditions in a given amount of time. “Hybridization efficiency” is a particular sub-class of binding efficiency, and refers to binding efficiency in the case where the binding components are polynucleotides.

It will also be appreciated that throughout the present application, that words such as “first”, “second” are used in a relative sense only. A “set” may have one type of member or multiple different types. “Fluid” is used herein to reference a liquid. The terms “symmetric” and “symmetrized’ refer to the situation in which the tunneling barriers from each electrode to the biopolymer are substantially equal in magnitude. The term “nanopore” refers to a pore or hole having a minimum diameter on the order of nanometers and extending through a thin substrate. Nanopores can vary in size and can range from 1 nm to around 300 nm in diameter. Most effective nanopores have been roughly around 1.5 nm to 30 nm in diameter. The thickness of the substrate through which the nanopore extends can range from 1 nm to around 700 nm.

The terms “translocation” and “translocate” refer to movement through a nanopore from one side of the substrate to the other, the movement occurring in a defined direction.

The terms “portion” and “portion of a biopolymer” refer to a part, subunit, monomeric unit, portion of a monomeric unit, atom, portion of an atom, cluster of atoms, charge or charged unit.

The term “adjacent” refers to anything that is near, next to or adjoining. For instance, a nanopore referred to as “adjacent to an excitable molecule” may be near an excitable molecule, it may be next to the excitable molecule, it may pass through an excitable molecule or it may be adjoining the excitable molecule. “Adjacent” can refer to spacing in linear, two-dimensional and three-dimensional space.

### The Fluorescence Process

Fluorescence is the result of a three-stage process that occurs in certain molecules (generally polyaromatic hydrocarbons or heterocycles) called fluorophores or fluorescent dyes. A fluorescent probe is a fluorophore designed to localize within a specific region of a biological specimen or to respond to a specific stimulus. The process responsible for the fluorescence of fluorescent probes and other fluorophores is often illustrated in simple stages.

Stage 1: Excitation

A photon of energy hν_{EX} is supplied by an external source such as an incandescent lamp or a laser and absorbed by the fluorophore, creating an excited electronic singlet state (S₁′). This process distinguishes fluorescence from chemiluminescence, in which the excited state is populated by a chemical reaction. For a molecule to phosphoresce, the excited singlet state produced by the absorption of radiation must change to the triplet state by intersystem crossing. This crossing involves a change in electron spin or a “forbidden” transition. In other words, the probability of this happening is low and the rate is slow. Therefore, in order to emit a photon and return to the ground state, the electron spin of an electron in the molecule must again change. Phosporescence lifetimes are, therefore, much longer than fluorescence lifetimes.

Stage 2: Excited-State Lifetime

The excited state exists for a finite time (typically 1-10 nanoseconds). During this time, the fluorophore undergoes conformational changes and is also subject to a multitude of possible interactions with its molecular environment. These processes have two important consequences. First, the energy of S₁′ is partially dissipated, yielding a relaxed singlet excited state (S₁) from which fluorescence emission originates. Second, not all the molecules initially excited by absorption (Stage 1) return to the ground state (S₀) by fluorescence emission. Other processes such as collisional quenching, Fluorescence Resonance Energy Transfer (FRET) and intersystem crossing (see below) may also depopulate S₁. The fluorescence quantum yield, which is the ratio of the number of fluorescence photons emitted (Stage 3) to the number of photons absorbed (Stage 1), is a measure of the relative extent to which these processes occur.

Stage 3: Fluorescence Emission

A photon of energy hν_{EM} is emitted, returning the fluorophore to its ground state S₀. Due to energy dissipation during the excited-state lifetime, the energy of this photon is lower, and, therefore, of longer wavelength, than the excitation photon hν_{EX}. The difference in energy or wavelength represented by (hν_{EX}−hν_{EM}) is called the Stokes shift. The Stokes shift is fundamental to the sensitivity of fluorescence techniques because it allows emission photons to be detected against a low background, isolated from excitation photons. In contrast, absorption spectrophotometry requires measurement of transmitted light relative to high incident light levels at the same wavelength. The entire fluorescence process is cyclical. Unless the fluorophore is irreversibly destroyed in the excited state (an important phenomenon known as photobleaching, see below), the same fluorophore can be repeatedly excited and detected. The fact that a single fluorophore can generate many thousands of detectable photons is fundamental to the high sensitivity of fluorescence detection techniques. With few exceptions, the fluorescence excitation spectrum of a single fluorophore species in dilute solution is identical to its absorption spectrum. Under the same conditions, the fluorescence emission spectrum is independent of the excitation wavelength, due to the partial dissipation of excitation energy during the excited-state lifetime. The emission intensity is proportional to the amplitude of the fluorescence excitation spectrum at the excitation wavelength.

Fluorescence Signals:

Fluorescence intensity is quantitatively dependent on the same parameters as absorbance—defined by the Beer-Lambert law as the product of the molar extinction coefficient, optical path length and solute concentration—as well as on the fluorescence quantum yield of the dye and the excitation source intensity and fluorescence collection efficiency of the instrument. In dilute solutions or suspensions, fluorescence intensity is linearly proportional to these parameters. When sample absorbance exceeds about 0.05 in a 1 cm pathlength, the relationship becomes nonlinear and measurements may be distorted by artifacts such as self-absorption and the inner-filter effect.

Background Fluorescence:

Fluorescence detection sensitivity is severely compromised by background signals, which may originate from endogenous sample constituents (referred to as autofluorescence) or from unbound or nonspecifically bound probes (referred to as reagent background). Detection of autofluorescence can be minimized either by selecting filters that reduce the transmission of E2 relative to E1 or by selecting probes that absorb and emit at longer wavelengths. Although narrowing the fluorescence detection bandwidth increases the resolution of E1 and E2, it also compromises the overall fluorescence intensity detected. Signal distortion caused by autofluorescence of cells, tissues and biological fluids is most readily minimized by using probes that can be excited at >500 nm. Furthermore, at longer wavelengths, light scattering by dense media such as tissues is much reduced, resulting in greater penetration of the excitation light.

Photobleaching:

Under high-intensity illumination conditions, the irreversible destruction or photobleaching of the excited fluorophore becomes the factor limiting fluorescence detectability. The multiple photochemical reaction pathways responsible for photobleaching of fluorescein have been investigated and described in considerable detail. Some pathways include reactions between adjacent dye molecules, making the process considerably more complex in labeled biological specimens than in dilute solutions of free dye. In all cases, photobleaching originates from the triplet excited state, which is created from the singlet state (S₁) via an excited-state process called intersystem crossing. The most effective remedy for photobleaching is to maximize detection sensitivity, which allows the excitation intensity to be reduced. Photobleaching rates are dependent to some extent on the fluorophore's environment.

Fluorophore-Fluorophore Interactions:

Fluorescence quenching can be defined as a bimolecular process that reduces the fluorescence quantum yield without changing the fluorescence emission spectrum; it can result from transient excited-state interactions (collisional quenching) or from formation of nonfluorescent ground-state species. Self-quenching is the quenching of one fluorophore by another; it therefore tends to occur when high loading concentrations or labeling densities are used. Studies of the self-quenching of carboxyfluorescein show that the mechanism involves energy transfer to nonfluorescent dimers.

Fluorescence resonance energy transfer (FRET) is a strongly distance-dependent excited-state interaction in which emission of one fluorophore is coupled to the excitation of another. Some excited fluorophores interact to form excimers, which are excited-state dimers that exhibit altered emission spectra. Excimer formation by the polyaromatic hydrocarbon pyrene is described elsewhere. Because they all depend on the interaction of adjacent fluorophores, self-quenching, FRET and excimer formation can be exploited for monitoring a wide array of molecular assembly or fragmentation processes such as membrane fusion, nucleic acid hybridization, ligand-receptor binding and polypeptide hydrolysis.

Other Environmental Factors:

Many other environmental factors exert influences on fluorescence properties. The three most common are: Solvent polarity (solvent in this context includes interior regions of cells, proteins, membranes and other biomolecular structures). Proximity and concentrations of quenching species, pH of the aqueous medium Fluorescence spectra may be strongly dependent on solvent. This characteristic is most often observed with fluorophores that have large excited-state dipole moments, resulting in fluorescence spectral shifts to longer wavelengths in polar solvents. Representative fluorophores include the aminonaphthalenes such as prodan, badan and dansyl, which are effective probes of environmental polarity in, for example, a protein's interior. Binding of a probe to its target can dramatically affect its fluorescence quantum yield (Monitoring Protein-Folding Processes with Anilinonaphthalenesulfonate Dyes). Probes that have a high fluorescence quantum yield when bound to a particular target but are otherwise effectively nonfluorescent yield extremely low reagent background signals (see above). Molecular Probes' ultrasensitive SYBR Green, SYBR Gold, SYTO, PicoGreen, RiboGreen and OliGreen nucleic acid stains are prime examples of this strategy. Similarly, fluorogenic enzyme substrates, which are nonfluorescent or have only short-wavelength emission until they are converted to fluorescent products by enzymatic cleavage (see below), allow sensitive detection of enzymatic activity.

Extrinsic quenchers, the most ubiquitous of which are paramagnetic species such as O₂ and heavy atoms such as iodide, reduce fluorescence quantum yields in a concentration-dependent manner. If quenching is caused by collisional interactions, as is usually the case, information on the proximity of the fluorophore and quencher and their mutual diffusion rate can be derived. This quenching effect has been used productively to measure chloride-ion flux in cells. Many fluorophores are also quenched by proteins. Examples are NBD, fluorescein and BODIPY dyes, in which the effect is apparently due to charge-transfer interactions with aromatic amino acid residues. Consequently, antibodies raised against these fluorophores are effective and highly specific fluorescence quenchers. Fluorophores such as BCECF and carboxy SNARF-1 that have strongly pH-dependent absorption and fluorescence characteristics can be used as physiological pH indicators. Fluorescein and hydroxycoumarins (umbelliferones) are further examples of this type of fluorophore. Structurally, pH sensitivity is due to a reconfiguration of the fluorophore's π-electron system that occurs upon protonation. Molecular Probes' BODIPY FL fluorophore and the Alexa Fluor 488 dye, both of which lack protolytically ionizable substituents, provide spectrally equivalent alternatives to fluorescein for applications requiring a pH-insensitive probe.

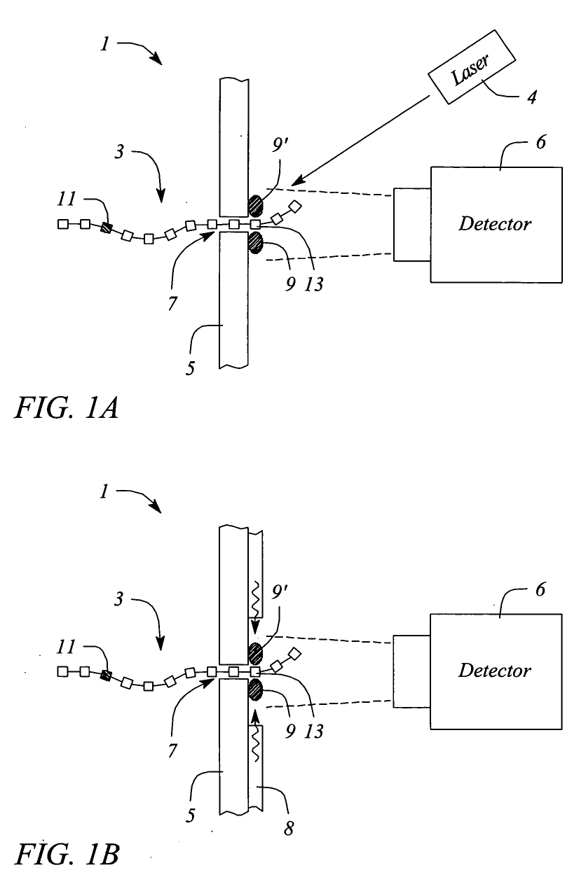

Referring now to FIGS. 1-3, the present invention provides a nanopore structure 1 for sequencing a biopolymer 3. The nanopore structure 1 comprises a substrate 5 having at least one nanopore 7 for sequencing the biopolymer 3. The nanopore system 1 comprises an excitable molecule 9 which may be positioned adjacent to the nanopore 7. The biopolymer 3 may comprise one or more quencher molecules 11 that quench a first excitation signal produced by the excitable molecule 9 after it has been irradiated by a light source 4. Modulations of the first excitation signal are detected by a detector 6 as the bipolymer 3 is translocated through the nanopore 7 in the substrate 5. Modulations of the first excitation signal are produced by the presence of one or more quencher molecules 11 present on the biopolymer 3.

A nanoscale moiety such as a biopolymer 3 is schematically depicted as a string of beads that is threaded through nanopore 7. The biopolymer 3 typically resides in an ionic solvent such as aqueous potassium chloride, not shown, which also extends through nanopore 7. It should be appreciated that, due to Brownian motion if nothing else, biopolymer 3 is always in motion, and such motion will result in a time-varying position of each bead within the nanopore 7. The motion of biopolymer 3 will typically be biased in one direction or another through the pore by providing an external driving force, for example by establishing an electric field through the pore between a set of electrodes.

Bead 13 is located near the mid-plane between first excitable molecule 9 and second excitable molecule 9′. If it is not in such a favorable position at one instant, the combination of Brownian motion and biased motion will ensure that it has been in such a favorable position immediately beforehand, or that it will be in such a favorable position immediately afterward. In addition, at the instant when bead 13 is in the desired favorable position, the two beads adjacent to bead 13 will not be in the desired favorable position. The use of additional excitable molecules associated with nanopore 7 is within the scope of the invention.

The biopolymer 3 may comprise a variety of shapes, sizes and materials. The shape or size of the molecule is not important, but it must be capable of translocation through the nanopore 7. For instance, both single stranded and double stranded RNA and DNA may be used as a biopolymer 3. In addition, the biopolymer 3 may contain groups or functional groups that are charged. Furthermore, metals or materials may be added, doped or intercalated within the biopolymer 3 to provide a net dipole, to provide a net charge, to provide conductivity through the biomolecule or to provide some combination of the above properties. Biopolymer 3 may comprise one or more quencher molecules 11 that quench the fluorescence or excitations signals of the excitable molecules 9 and/or 9′. It should be noted that the quencher molecule 11 may comprise a portion of biopolymer 3, may be attached to biopolymer 3 or may be positioned adjacent to the biopolymer 3 it is attached or associated with. In each case the quencher molecule 11 identifies the presence or absence of a particular base, nucleotide, peptide or monomer unit of the biopolymer 3.

The optional substrate 5 may comprise a variety of materials known in the art for designing substrates and nanopores. Substrate 5 may comprise one or more layers of one or more materials including, but not limited to, membranes, edgeids, silicon nitride, silicon dioxide, platinum or other metals, silicon oxynitride, silicon rich nitride, organic polymers, and other insulating layers, carbon based materials, plastics, metals, or other materials known in the art for etching or fabricating semiconductor or electrically conducting materials. Substrate 5 need not be of uniform thickness. Substrate 5 may or may not be a solid material, and for example, may comprise in part or in whole a edgeid bilayer, a mesh, wire, or other material in which a nanopore may be constructed. Substrate 5 may comprise various shapes and sizes. However, it must be large enough and of sufficient width to be capable of forming the nanopore 7 through it.

The nanopore 7 may be positioned anywhere on/through the substrate 5. The nanopore 7 may also be established by the spacing between the first excitable molecule 9 and the second excitable molecule 9′. The nanopore 7 may range in size from 1 nm to as large as 300 nm. In most cases, effective nanopores for sensing or characterizing biopolymers would be in the range of around 2-20 nm. These size nanopores are just large enough to allow for translocation of a biopolymer 3. The nanopore 7 may be established using any methods well known in the art. For instance, the nanopore 7 may be sculpted in the substrate 5 by way of low-energy argon ion beam sculpting of an initially larger hole formed by etching or focused ion beam machining, or by sputtering, etching, photolithography, or other methods and techniques well known in the art.

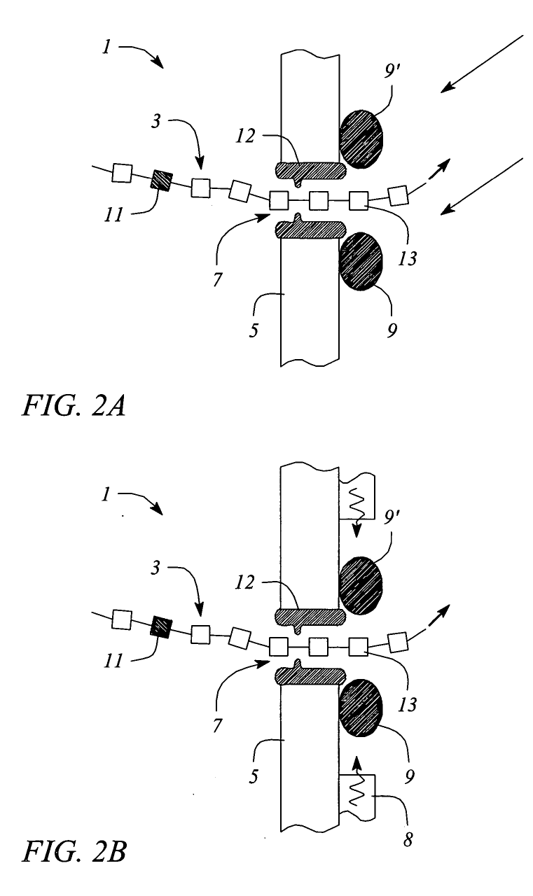

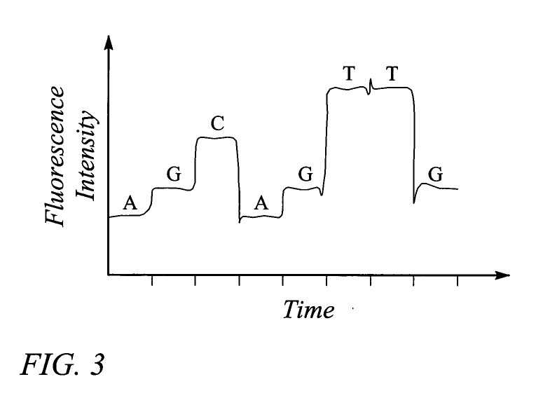

Referring now to FIGS. 1A-2B, various embodiments of the present invention can be seen. FIG. 1A shows the substrate 5 comprising a nanopore 7 with the biopolymer 3 translocating through the nanopore 7. Excitable molecules 9 and 9′ are positioned on one side of the substrate 5. The positioning of these molecules may also be near the entrance of the nanopore 7 as opposed to the exit as shown in the diagram. In fact, the excitable molecules 9 and 9′ may be positioned anywhere adjacent to the nanopore 7. It is important to the invention that the excitable molecules 9 and/or 9′ be placed in close proximity or near to the nanopore 7 so that the excitation signal (i.e. fluorescence of the exitable molecule) may be affected or modulated by the approach or presence of one or more quencher molecules 11. In this embodiment of the invention a light source 4 is employed in conjunction with the detector 6. The light source 4 irradiates the excitable molecules 9 and/or 9′. Concomitantly, the biopolymer 3 is translocated through the nanopore 3 (in the diagram this is from the left side of the nanopore to the right side having the detector). The detector 6 is designed for detecting any changes in overall fluorescence output. For instance, there may be constant fluorescence or phosphorescence from the continual or pulsed irradiation of the excitable molecules 9 or 9′. However, when a quencher molecule 11 is moved into the appropriate position 13 in the nanopore 7, the overall signal to the detector 6 is lessened or eliminated. These fluctuations in fluorescence are determined by the detector 6. There are any number of ways of detecting such fluctuations. For instance, additional hardware, software or a combination of both may be employed with the detector 6. A background level or maximum intensity can be calibrated during the full irradiation of the excitable molecules 9 and/or 9′. Comparisons can then be made by taking snap shots or micro spectra over time. Fluctuations can then be stored and compared. FIG. 3 shows a theoretical stochastic sensing pattern that may be obtained using such a technique. An important characteristic of the invention is for the detector to detect changes in overall fluorescence or modulation of the excitable molecules that are being irradiated by the light source 4. The various effects by these quenchers on the excitable molecules determine the overall line shape or intensity level recorded in the final spectrum. For instance, FIG. 3 shows how A, G, an C may differ in the diagram for adenine (A), guanine (G) and cytosine (C) present in a biopolymer 3 that has translocated through the nanopore 7.

Although the invention shows the dual application of excitable molecules 9 and 9′, it is within the scope of the invention that multiple quencher molecule(s) and/or excitable molecules may be employed. The excitable molecules may be placed anywhere adjacent to the nanopore 7 and may also be placed on opposing sides of the nanopore 7. In addition, the light source 4 may be used to irradiate the excitable molecules in a sequential manner or concomitantly. Also, it is within the scope of the invention the multiple light sources may be employed on both the entrance of the nanopore 7 and/or the exit of the nanopore 7.

FIG. 1B shows a similar embodiment as described above in FIG. 1A. However, in this embodiment of the invention, a light pipe 8 is employed to conduct light from a light source 4 to irradiate one or more adjacent excitable molecules 9 and/or 9′. As described above, the light pipe 8 may be pulsed or designed to provide continual irradiation.

FIGS. 2A-2B show similar embodiments to the embodiments as described in FIGS. 1A and 1B. However, in these embodiments the nanopore 7 comprises a pore forming agent 12 such as α-hemolysin. The pore forming agent 12 is employed to define the nanopore 7. Other pore forming agents 12 may be employed that comprise biological and non-biological material. In each case, the pore forming agent 12 must be large enough for the biopolymer 3 to translocate across the substrate 5 and allow for the sequencing and detection of the base units or monomers of the biopolymer 3.

Having now described the apparatus of the present invention, a description of the method is now in order.

The present invention also provides a method for sequencing and characterizing a nanoscale moiety moved through the nanopore 7 of the substrate 5. The method for characterizing the nanoscale moiety comprises providing a substrate having an excitable molecule adjacent to a nanopore and moving a portion of a nanoscale moiety with an attached quencher molecule past the excitable molecule that produces a first excitation signal to quench the excitable molecule and produce a modulation in the first excitation signal that is indicative of the presence or absence of a particular monomeric unit of the nanoscale moiety. The method for sensing a portion of a nanoscale moiety, comprises providing a substrate having an excitable molecule adjacent to a nanopore, exciting the excitable molecule to produce a first excitation signal, and moving a portion of the nanoscale moiety with a quencher molecule past the excitable molecule to quench or modulate the first excitation signal and determine the presence of the quencher molecule or portion of a molecule associated with the quencher molecule.

1. A nanopore structure with nanopore for sensing a nanoscale moiety having a quencher molecule, comprising:
(a) a substrate having a nanopore;
(b) an excitable molecule attached to the substrate adjacent to the nanopore;
(c) a light source for exciting the excitable molecule attached to the substrate adjacent to the nanopore, said light source for producing a first excitation signal from said excitable molecule that may be modulated by a quencher molecule comprising a portion of said biopolymer as said biopolymer translocates through said nanopore of said substrate; and
(d) a detector adjacent to said substrate and nanopore for detecting the signal modulation changes of said excitable molecule as said quencher molecules are moved adjacent to said excitable molecules.

2. A nanopore structure as recited in claim 1, wherein the light source comprises a laser.

3. A nanopore structure as recited in claim 1, wherein the light source comprises a light pipe.

4. A nanopore structure as recited in claim 1, wherein the excitable molecule on the is selected from the group consisting of an ion, a monomer, an atom, a metal, a halide, an amino acid, a nucleotide, a simple sugar, a quantum dot, and a nanosphere.

5. A nanopore structure as recited in claim 1, wherein the nanoscale moiety comprises a biopolymer.

6. A nanopore structure as recited in claim 3, wherein the biopolymer is selected from the group consisting of a polypeptide, a polynucleotide, a synthetic polymer, a non synthetic polymer and a polysaccharide.

7. A nanopore structure as recited in claim 4, wherein the polynucleotide is selected from the group consisting of deoxyribonucleic acid (DNA), ribonucleic acid (RNA), single stranded DNA, single stranded RNA, double stranded RNA, double stranded DNA, DNA complexed to RNA, DNA bound to protein, RNA bound to protein, transfer RNA (tRNA), and messenger RNA (mRNA).

8. A nanopore structure as recited in claim 1, wherein the nanopore is from 1 nanometer to 10 nanometers in diameter.

9. A nanopore structure as recited in claim 1, wherein the excitable molecule comprises a chromophore.

10. A nanopore structure as recited in claim 1, wherein the excitable molecule comprises a fluorophore.

11. A nanopore structure as recited in claim 9, wherein the fluorophore is selected from the group consisting of an aromatic amino acid, a nucleic acid, a derivatized nucleic acid, fluorescein, a quantum dot and coumarin.

12. A nanopore structure as recited in claim 1, wherein the quencher molecule is selected from the group consisting of cesium chloride, potassium iodide, quinaldic acid, acrylamide, pyridine, 8-anilinonaphthalene-1-sulfonate (ANS),

13. A method for sensing a portion of a nanoscale moiety, comprising:
(a) providing a substrate having an excitable molecule adjacent to a nanopore; and
(b) moving a portion of a nanoscale moiety with a quencher molecule past the excitable molecule to quench the excitable molecule and determine the identity of the portion of the nanoscale moiety.

14. A method for sensing a portion of a nanoscale moiety, comprising:
(a) providing a substrate having an excitable molecule adjacent to a nanopore;
(b) exciting the excitable molecule;
(c) moving a portion of the nanoscale moiety with a quencher molecule past the excitable molecule to quench the quencher molecule and determine the identity of the portion of the nanoscale moiety.
